(19) United States
(12) Patent Application Publication
(10) Pub. No.: US 2014/0343041 A1
(43) Pub. Date: Nov. 20, 2014

(54) Topical Ophthalmic Formulations for the Treatment and Prevention of Migraine Headache

(71) Applicant: Ora, Inc., Andover, MA (US)

(72) Inventors: Matthew J. Chapin, Amesbury, MA (US); Richard Abelson, Gilbert, AZ (US)

(21) Appl. No.: 14/209,346

(22) Filed: Mar. 13, 2014

Related U.S. Application Data
(60) Provisional application No. 61/788,702, filed on Mar. 15, 2013.

(51) Int. Cl. A61K 31/4535; A61K 31/5377; A61K 9/00; A61K 45/06

(52) U.S. Cl. CPC A61K 31/4535; A61K 45/06; A61K 31/5377; A61K 9/0048
USPC 514/214.02; 514/324; 514/236.2

(57) ABSTRACT

The present invention provides topical ophthalmic formulations comprising a combination of one or more antihistamine agents and optionally one or more vasculature modifying agents such as a β adrenergic receptor antagonist. Also provided are methods of using the formulations of the invention for treating and/or preventing symptoms associated with migraine headache, and for reducing the frequency, severity and duration of migraine attacks.

### CROSS REFERENCE TO RELATED APPLICATIONS

The present application claims priority to U.S. Provisional Application No. 61/788,702, filed on Mar. 15, 2013, the entire disclosure which is incorporated herein by reference in its entirety.

### BACKGROUND

Migraine is a paroxysmal disorder characterized by recurrent attacks of headache, with or without associated visual and gastrointestinal disturbances. The International Society of Headache classifies headaches as primary, secondary or cranial neuralgias. There are 4 categories of primary headaches according to the IHS's International Classification of Headache Disorders which are Migraine, Tension-Type Headache, Cluster Headache, & Other primary headaches. Secondary headaches are diagnosed in addition to a primary headache to provide a better course of treatment for the patient. Migraine headaches are diagnosed as one of two headache disorders. The first disorder, common migraine, typically includes headaches with unilateral location, pulsating quality & moderate or severe intensity. Additionally, common migraines are associated with either nausea/vomiting or photo/phonophobia but sometimes both. The second syndrome, classic migraine, features the same symptoms of the common migraine but also includes a visual aura which is typically associated with fully reversible visual/sensory symptoms & fully reversible dysphasic speech disturbance. Migraine, with or without aura, is a remarkably common condition and prevalence among Caucasians is in the range of 4 to 6 percent among men and 13 to 18 percent among women. Migraine sufferers or ‘migraineurs’ will occasionally present migraine symptoms that will resolve after treatment of another underlying condition e.g. headache attributed to disorder of the eye or headache attributed to rhinosinusitus. These types of headaches are classified by the IHS' ICHD as ‘Secondary Headaches.’ While there are a plethora of secondary headache classifications, many migraineurs suffer from headaches that are strictly associated with primary headache.

The mainstay of pharmacologic therapy is the judicious use of one or more of the many drugs that are effective in the treatment of primary headaches such as migraine. The major pharmacologic classes of drugs that have historically been effective in treatment of migraine are anti-inflammatory agents, serotonin receptor agonists, 5-hydroxytryptamine (5-HT) agonists, and dopamine receptor antagonists. Acute attack medications, particularly codeine or barbiturate-containing compound analgesics, have a propensity to aggravate headache frequency and induce a state of refractory daily or near-daily headache classified as medication-overuse headache. This condition is likely not a separate headache entity but a reaction of the migraine patient to a particular medicine. Migraine patients who have two or more headache days a week are cautioned about frequent analgesic use. Patients with an increasing frequency of migraine attacks or with attacks that are either unresponsive or poorly responsive to abortive treatments should be considered for preventive treatment. Drug classes currently utilized for migraine prophylaxis include β adrenergic receptor antagonists ((β blockers), calcium channel blockers, anticonvulsants, anti-depressants, skeletal muscle relaxants, caffeine/anti-inflammatory combinations and even botulinim toxin (Botox). With the exception of Botox injections, drugs used in migraine prophylaxis must be taken daily, and there is usually a lag of at least 2-12 weeks before an effect is seen. Significant side effects including but not limited to cardiac disorders, renal failure, cognitive-related dysfunction, behavioral disturbance and somnolence/fatigue are associated with the use of many of these agents. Current migraine therapy is typically orally administered, absorption of which is compromised in patients with migraine due to gastric stasis, nausea and vomiting commonly present during the migraine attack. Thus, there is a need for a more effective therapy for treating and preventing migraine headaches. The present invention meets this need and other needs.

### SUMMARY OF THE INVENTION

An embodiment of the invention is a topical ophthalmic formulation including at least one anti-histamine agent in an amount effective to treat or prevent the signs and symptoms of migraine headache.

In a related embodiment, the antihistamine agent is a mast cell stabilizer. For example, the anti-histamine agent (or anti-histamine/mast cell stabilizer) is ketotifen fumarate, epinastine, bepotastine, cetirizine, olopatadine hydrochloride, alcaftadine, norketotifen, azelastine chlorpheniramine, brompheniramine, diphenhydramine, clemastine, hydroxyzine, chlorpheniramine, doxylamine, alimemazine, phenyltoloxamine, promethazine, mepyramine, cyproheptadine, hydroxyzine, antazoline, tripelennamine, orphenadrine, bromazine, clemastine, dimetindene, azatadine, loratadine, fexofenadine, desloratadine, levocetirizine, carbinoxamine, triprolidine, dexchlorpheniramine, pheniramine, ebastine, dexbrompheniramine, astemizole, rupatadine, mizolastine, acrivastine, bilastine, terfenadine, quifenadine, or levocabastine; derivatives thereof; or pharmacologically active salts thereof. For example, the concentration of the ketotifen is about 0.025-0.05%.

In another related embodiment, the anti-histamine/mast cell stabilizer is alcaftadine. For example, the concentration of alcaftadine is about 0.025-0.5%.

According to other embodiments, the migraine headache is with or without aura. In related embodiments the ophthalmic formulation is such that when used in an effective amount for treatment the frequency, severity and duration of migraine headache is reduced.

In one aspect the ophthalmic formulation further includes one or more vasculature modifying agents. For example, the vasculature modifying agent is a β adrenergic receptor antagonist such as an H-1 antagonist. In related embodiments the H-1 antagonist is bisoprolol, timolol, propranolol, nadolol, metoprolol, metipranolol, nebivolol, or betaxolol.

In a related embodiment, the antihistamine agent and the β adrenergic receptor antagonist of the ophthalmic formulation are, respectively, ketotifen fumarate and timolol maleate. For example, the concentration of ketotifen fumarate is about 0.025-0.05%, and the concentration of timolol maleate is about 0.25-0.5%. Alternatively, the antihistamine is alcaftadine and the β adrenergic receptor antagonist is timolol maleate. For example, the concentration of alcaftadine is about 0.025-0.5% and the concentration of timolol maleate is 0.25-0.5%.

Another aspect the invention is a method for treating and/or preventing the signs and symptoms of migraine headache including administering the topical ophthalmic formulation of any of the embodiments above. In related embodiments, the antihistamine is ketotifen fumarate and the β adrenergic receptor antagonist is timolol maleate. For example, the concentration of ketotifen fumarate is about 0.025-0.05% and the concentration of timolol maleate is about 0.25-0.5%. Alternatively, the antihistamine is alcaftadine and the β adrenergic receptor antagonist is timolol maleate. For example, the concentration of alcaftadine is about 0.025-0.5% and the concentration of timolol maleate is about 0.25-0.5%.

### DETAILED DESCRIPTION

The present invention is based on unexpected findings that topical ophthalmic formulations of anti-histamines are useful for treating and preventing migraine headaches. The prophylactic treatments of migraine headaches, in agreement with the present invention, can reduce the frequency of migraine attacks, as well as reduce their severity and duration when they do occur. The topical ophthalmic formulations include one or more anti-histamine agent (e.g., a dual-acting antihistamine/mast cell stabilizer agent), and optionally include other vasculature modifying agents (such as but not limited to: beta-blockers and other adrenergic agents) in amounts which are effective to achieve the desired purpose of preventing migraine headaches or reducing the severity or duration of an attack of migraine headache once it has occurred.

For convenience, before further description of the present invention, certain terms employed in the specification, examples, and appended claims are defined below. These definitions should be read in light of the remainder of the disclosure and understood as by a person of ordinary skill in the art.

The term “aqueous” typically denotes an aqueous composition wherein the carrier is to an extent of >50%, more preferably >75% and in particular >90% by weight water.

The term “migraine” refers to a familial disorder characterized by periodic, commonly unilateral, often pulsatile headaches that begin in childhood, adolescence, or early adult life and recur with diminishing frequency during advancing years. Two closely related clinical syndromes have been identified, the first called migraine with aura and the second, migraine without aura (terminology of the International Headache Society's International Classification of Headache Disorders). Migraine without aura, previously referred to as common migraine is classified as at least 5 headache attacks lasting anywhere between 4-72 hours where the headache has at least two of following qualities: unilateral location of pain, pulsating quality of pain, moderate to severe pain intensity which can be aggravated by, or cause the avoidance of routine physical activity. Additionally, migraine without aura is associated with either nausea and or vomiting & photo/phonophobia but sometimes both. Migraine with aura, the term now used to denote classic migraine, is classified as at least 2 headache attacks with aura which are not associated with motor weakness, and consists of at least one of the following: fully reversible visual symptoms, fully reversible sensory symptoms, and fully reversible dysphasic speech disturbance. Auras are also associated with two of the following: homonymous visual symptoms and/or unilateral sensory symptoms. At least one aura symptom develops over 5 minutes or longer with other symptoms developing in a similar fashion, and each symptom lasts between 5 minutes and an hour. Additionally, symptoms from common migraine must begin with the aura or be followed by the aura within 60 minutes of the onset of migraine, followed in a few minutes by hemicranial, or in about one-third of cases, by bilateral headache, nausea, and sometimes vomiting, all of which last for hours or as long as a day or two. As mentioned previously, the hemicranial and the throbbing (pulsating) aspects of migraine are its most characteristic features and each patient displays a tendency for the pain to affect one side or the other of the cranium, but not exclusively.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of migraine. The effective amount may vary depending on such factors as the disease or condition being treated, the particular composition being administered, or the severity of the disease or condition. One of ordinary skill in the art may empirically determine the effective amount of a particular agent without necessitating undue experimentation.

A “patient,” “subject,” or “host” to be treated by the subject method refers to either a human or non-human animal, such as primates, mammals, and vertebrates.

The phrase “pharmaceutically acceptable” is art-recognized and refers to compositions, polymers and other materials and/or salts thereof and/or dosage forms which, within the scope of sound medical judgment, are suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

The term “pharmaceutically acceptable salts” is art-recognized, and refers to relatively non-toxic, inorganic and organic acid added to the compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl) aminoethane; and the like. See, for example, J. Pharm. Sci., 66:1-19 (1977). The term “preventing,” when used in relation to a condition, such as migraine headache, is art-recognized, and refers to administration of a composition, which reduces the frequency of, or delays the onset of, signs and/or symptoms of a medical condition in a subject relative to a subject who has the medical condition and is not receiving the composition.

The term “treating” is an art-recognized term, which refers to curing as well as toameliorating at least one symptom of any condition or disease.

### Role of Histamines in Migraine Etiology

Histamine is a biologically active amine that functions as a neurotransmitter, is found in non-neural tissues, has complex physiologic and pathologic effects through multiple receptor subtypes, and is often released locally. Histamine, along with serotonin, and other endogenous peptides such as prostaglandins and leukotrienes, and cytokines, are called autacoids (Greek, “self-remedy”) or local hormones in recognition of their properties (Lange Pharmacology 10^{th} edition). Histamine exerts its biologic actions by binding to specific cellular receptors located on the surface of the cell membrane.

Anti-histamines are compounds that antagonize the action of histamine and are of considerable clinical usefulness. The four different clinically relevant histamine receptors characterized thus far are designated H₁ to H₋₄. H₁ receptor antagonists block the actions of histamine by reversible competitive antagonism at the H₁ receptor.

Clinical evidence suggesting the involvement of histamine in migraine is based on the findings showing elevated histamine levels in serum during migraine attacks (Heitley et al., 1982; Haimart et al., 1987). Additionally, histamine administration induced migraine headache more often in migraineurs than in control subjects, and it has been demonstrated that patients with allergic rhinitis were 14.3 times more likely to have a migraine headache than control subjects (Krabbe & Oleson, 1980; Ku et al. 2006). These studies also suggest that histamine plays a role in the pathogenesis of migraine headaches. Despite the fact that histamine has been implicated in the pathogenesis of migraine, numerous clinical studies using antihistamine treatments have failed to demonstrate efficacy in treating the severity of the symptoms of migraine (Mansfield 1990). Additionally, a post marketing study of the antihistamine levocetirizine, that used prescription-event marketing data revealed that headache/migraines were in fact positively associated with the first month of treatment (Layton D et al. 2011).

Preclinical evidence suggests the involvement of mast cells in the pathogenesis of migraine as mast cells have been linked to neurogenic meningeal inflammation associated with migraine (Zhang et al. 2007). The Dura mater of the meninges is most heavily innervated by pain fibres (meningeal nociceptors) and is also populated by resident mast cells in both humans and rodents. Dural mast cells because of their proinflammatory properties have been suggested to play a role in migraine headaches. In fact, mast cell degranulation has been demonstrated to excite meningeal nociceptors, promote pERK (a marker for nociceptor activation in CGRP-positive dural fibers) expression in meningeal nociceptors, and to activate nociceptive trigeminovascular brainstem neurons in vivo (Levy et al., 2007). Additionally, mast cells residing near the blood vessels and meningeal nociceptive fibres have the capacity to initiate or amplify inflammatory responses by releasing histamine in addition to other mediators like serotonin, cytokines, leukotrienes, prostaglandins etc. (Ba'albaki & Rapoport, 2008, Theoharides et al. 2005) It has been suggested that in conditions where mast cells are activated during migraine attack, histamine may be one of the mediators playing a role in promoting migraine headache. Prophylactic effect of antihistamines has been shown in migraine patients (Lewis et al., 2004; Togha et al., 2006, Lewis et al. 2008). Yet, none of these studies featured antihistamines that were administered via a topical ophthalmic solution, reemphasizing the unexpected nature of the invention to one of ordinary skill in the art.

### β-Blockers and Migraine Therapy

A β adrenergic receptor antagonist (beta-blocker) is any compound that blocks the biological activity of beta-adrenergic receptor by binding to the receptor without eliciting the biological response normally stimulated by receptor agonist. Receptor antagonism can be competitive, when the antagonist competes directly with the agonist at the receptor's ligand binding site, or it can be non-competitive. There are several different subtypes of beta adrenergic receptors; beta-1, beta-2, and beta-3. Beta-blockers can be non-selective or selective antagonists of beta-adrenergic receptors. For example, without limitation, Propranolol is a competitive non-selective beta-blocker, which is considered as a prototype to which other beta-blockers are compared.

Recent studies indicate that the use of beta-blockers has a place in migraine prophylaxis protocol. In a case report, a 64 year old woman with history of common migraine did not experience any further attacks of migraine after she was started on topical timolol maleate 0.5% for bilateral ocular hypertension (Bhagey et al. 2004). In a case control study (Yarangümeli et al., 2003), a group of subjects receiving a topical beta-blocker, betaxolol, showed improvement in their migraine related complaints. Additionally, in a study conducted using twice daily dosing with topical timolol maleate 0.5% ophthalmic solution for 8 days, the peak plasma concentration of 0.5 ng/ml was seen within 4 hours following first dose (Shedden et al., 2001).

### Pharmaceutical Compositions

The invention features novel topical ophthalmic formulations comprising an effective amount of one or more antihistamine agents (e.g., a mast cell stabilizer), optionally in combination with one or more vasculature modifying agents, in a pharmaceutically acceptable carrier for the treatment and prevention of migraine headaches. Surprisingly, the use of topical ophthalmic antihistamines has the ability to treat and prevent signs and symptoms of migraine headache and to reduce the frequency, severity, and duration of migraine attacks. Topical ophthalmic administration of antihistamines alone or in combination with a vasculature modifying agent has never been previously contemplated for the treatment of migraine by one of ordinary skill in the art despite the previously reported evidence for the method of action of a stand-alone mast cell stabilizing agent .Without intending to be bound by any theory, the ophthalmic formulations of the invention treat or prevent migraine headache by targeting pain pathways arising in the trigeminal meningeal pain receptors (nociceptors) and trigeminovascular brainstem neurons as mentioned previously. The antihistamine component will control the release of inflammatory mediators that are involved in the painful neurogenic vasodilatation of meningeal blood vessels that are the key component of migraine headache. Presumably, the antihistamine is a mast cell stabilizing agent by itself or in combination with a vasculature modifying agent, which can directly inhibit the degranulation of mast cells which have been directly implicated in the excitation of meningeal nociceptors and trigeminovascular brainstem neurons. The use of antihistamine agents will further help abort the attack once it has occurred as well as reduce the severity of the attack. This unexpected finding is confirmed by both of our n=2 studies.

The topical ophthalmic formulations of the invention provide a further advantage over previous therapeutic agents/treatment regimens for migraine headache that are orally administered in that absorption of the topical ophthalmic formulations of the invention is not compromised by the gastric stasis, nausea or vomiting that is commonly seen in patients with migraine. Zecuity, a recently approved topical dermal sumatriptan patch for the treatment of migraine, significantly reduced Migraine Related Nausea (MRN) compared to the placebo treatment (Medtrack 2013). Additionally, the topical ophthalmic formulation is significantly different from other topical treatments for migraine such as Zecuity and does not function via the same MOA as current systemic treatments for migraine. As a consequence of the different route of administration, topical ocular treatment also helps avoid the systemic side effects that are associated with oral or IV delivery routes due to the clearance of the topical treatment from the ocular surface. While there is a topical nasal SHT₂/H1 receptor antagonists treatment for migraine that is currently in phase 2 of development by Winston Pharmaceuticals Dolorac (Doxepin 0.4%), there are no ongoing clinical trials, and there is no reason to suggest that this treatment has a similar MOA to a topical ophthalmic solution.

The beta-blocker component in the topical ophthalmic formulation of the invention can be a subtype selective (i.e., beta-1, beta-2 and beta-3 adrenergic receptor) or a non-subtype selective beta-adrenergic receptor antagonist. Preferred beta-blockers are those that lack intrinsic sympathomimetic activity (ISA) i.e., partial agonist activity (Goodman and Gilman's Pharmacology, 11^{th} edition). Examples of beta-blockers that lack ISA and have previously been used for the preventive treatment of migraine, include, atenolol, metoprolol, nadolol, bisoprolol, timolol, betaxolol, derivatives or pharmacologically-active salts thereof.

Examples of non-subtype selective beta-adrenergic antagonists include, but are not limited to, propranolol, timolol, nadolol, derivatives thereof, and pharmacologically active salts thereof.

Examples of selective beta-1 adrenergic receptor antagonists include, but are not limited to metoprolol, atenolol, bisoprolol, esmolol, Betaxolol, derivatives thereof, and pharmacologically active salts thereof.

The antihistamine component in the topical ophthalmic formulation of the invention can be an H₁, H₂, H₃ or H₄ antagonist. The H₁ antagonists are conveniently divided into first-generation and second-generation agents. Examples of first generation antihistamines include, but are not limited to, chlorpheniramine, brompheniramine, diphenhydramine, clemastine, hydroxyzine, chlorpheniramine, doxylamine, alimemazine, phenyltoloxamine, promethazine, mepyramine, cyproheptadine, hydroxyzine, antazoline, tripelennamine, orphenadrine, bromazine, clemastine, dimetindene, azatadine, derivatives thereof, and pharmacologically active salts thereof. They are distinguished from the second-generation drugs by their relatively strong sedative effects. Second-generation non-sedating histamine H₁-receptor-blocking drugs include, but are not limited to loratadine, fexofenadine, desloratadine cetirizine, levocetirizine, carbinoxamine, triprolidine, ketotifen, dexchlorpheniramine, pheniramine, olopatadine, ebastine, dexbrompheniramine, astemizole, rupatadine, mizolastine, acrivastine, bilastine, bepotastine, terfenadine, quifenadine, azelastine, levocabastine, epinastine derivatives thereof, and pharmacologically active salts thereof.

In a certain embodiment, the antihistamine component in the ophthalmic formulations of the invention is an H₁ antagonist. Preferably, the H₁ antagonist is a mast cell stabilizer. Mast cell stabilizers are drugs that prevent the release of mediators from mast cells. Exemplary antihistamines with mast cell stabilizing properties include, but are not limited to, ketotifen fumarate, olopatadine hydrochloride, azelastine epinastine, alcaftadine, bepotastine, derivatives thereof, and pharmacologically active salts thereof.

Ketotifen is a selective H₁ histamine receptor antagonist with mast cell stabilizing properties. The advantage offered by dual acting antihistamines is the rapidity of symptomatic relief offered by immediate histamine receptor antagonism, coupled with long-term disease-modifying benefit of mast cell stabilization. In the United States, ketotifen fumarate ophthalmic drops (ZADITOR®; Novartis Ophthalmics), was approved by the FDA on Jul. 2, 1999 for the temporary prevention of itching of the eye due to allergic conjunctivitis (NDA 21-066).

The beta-blocker/mast cell stabilizer combinations in the ophthalmic formulations of the invention include combinations of any of the above-described beta-blockers and antihistamine/mast cell stabilizer agents. Exemplary beta-blocker/mast cell stabilizer combinations include, for example without limitation, a combination of timolol maleate and ketotifen fumarate, or of timolol maleate and alcaftadine; and a combination of timolol maleate; betaxolol; and ketotifen fumarate or alcaftadine.

Pharmaceutical ophthalmic formulations typically contain an effective amount, e.g., 0.001% to 10% wt/vol., preferably 0.005% to 5%, even more preferably 0.01% to 5% of an active agent. The amount of active ingredient will vary with the particular formulation and the disease state for which it is intended. Preferably, the effective amount of active agent present in the formulations of the invention should be sufficient to treat or prevent migraine headaches.

In yet another particular embodiment, the ketotifen fumarate is present in the ophthalmic formulation of the invention at a concentration of about 0.01% to 0.5% (w/v), or any specific value within said range. For example, ketotifen fumarate is present in a concentration of about 0.01%, 0.02%, 0.03%, 0.04%, 0.05%, 0.06%, 0.07%, 0.08%, 0.09%, 0.1%, 0.2%, 0.3%, 0.4% or 0.5% (w/v). For example, alcaftadine is present in a concentration of about 0.01%, 0.02%, 0.03%, 0.04%, 0.05%, 0.06%, 0.07%, 0.08%, 0.09%, 0.1%, 0.2%, 0.25%, 0.3%, 0.4% , 0.5%, or 1% (w/v).

The active agents of the may be in the form of a pharmaceutically acceptable salt.

The pharmaceutical compositions of the invention described above may additionally comprise other active ingredients, including, but not limited to, and vasoconstrictors, antiallergenic agents, anesthetics, analgesics, dry eye agents (e.g. secretagogues, mucomimetics, polymers, lipids, antioxidants), etc., or be administered in conjunction (simultaneously or sequentially) with pharmaceutical compositions comprising other active ingredients, including, but not limited to, and vasoconstrictors, antiallergenic agents, anesthetics, analgesics, dry eye agents (e.g. secretagogues, mucomimetics, polymers, lipids, antioxidants), etc.

Preferably, the pharmaceutical compositions according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be ointments, suspensions, emulsions, oils, viscous or semi-viscous gels, or other types of solid or semi-solid compositions.

Any of a variety of carriers may be used in the formulations of the present invention including water, mixtures of water and water-miscible solvents, such as C₁- to C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral carbopol, or mixtures of those polymers. The concentration of the carrier is, typically, from 1 to 100000 times the concentration of the active ingredient.

Additional ingredients that may be included in the formulation include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example, be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids, which is equivalent to a 0.9% solution of sodium chloride or a 2.5% solution of glycerol. An osmolality of about 225 to 400 mOsm/kg is preferred, more preferably 280 to 320 mOsm/kg.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N-(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenymercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sepazonium, sodium perborate, Purite(tm), sodium chlorite, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients, who wear contact lenses, or those who use several topical ophthalmic drops and/or those with an already compromised ocular surface (e.g. dry eye) wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol, polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

In embodiments wherein the formulation is an ointment, a preferred ointment base used to prepare the ophthalmic ointment of the present invention may be one that has been used in conventional ophthalmic ointments. In particular, the base may be liquid paraffin, white petrolatum, purified lanolin, gelatin hydrocarbon, polyethylene glycol, hydrophilic ointment base, white ointment base, absorptive ointment base, Macrogol (Trade Name) ointment base, simple ointment base, and the like.

The ophthalmic ointment may comprise further conventional excipients other than the ointment base in a suitable range that does not affect the intended functions and stability of the active ingredients. Examples of such excipients include antiseptics such as parahydroxybenzoate, chlorobutanol, benzalkonium chloride and the like; surfactants such as polysorbate 80, polyoxyl 40 stearate, polyoxyethylene hydrogenated castor oil, and the like; stabilizers such as sodium edetate, citric acid, and salts thereof; alcohols such as glycerol, lanolin alcohol, cetanol, and the like; esters such as isopropyl myristate, ethyl linoleate, and the like; and oils such as olive oil and triglycerides of middle-chained fatty acids.

### Methods of Use

The invention also features methods of treating and preventing the signs and symptoms of migraine headache in a subject, as well as methods for reducing the frequency, severity and duration of migraine headache in a subject, including use of the novel topical ophthalmic formulations described above. For example, a method of treating and/or preventing migraine headache, or for reducing the frequency, duration and severity of migraine headache may include administering to the eye surface of the subject in need thereof, a formulation comprising an effective amount of one or more beta-blockers and one or more antihistamine agents (e.g., a mast cell stabilizer), in a pharmaceutically acceptable carrier.

Migraine headaches that can be treated or prevented in accordance with the present invention include migraine with and without aura. In addition to reducing the frequency, severity and duration of headache, the methods of present invention can also reduce or eliminate the frequency, severity and duration of any of the associated symptoms associated with migraine headache including but not limited to the symptoms as listed on IHS's ICHD such as nausea, vomiting, sensitivity to light, lightheadedness, scalp tenderness, visual disturbances, vertigo, altered consciousness.

The effective amount of beta-blockers and antihistamine agent in the formulation will depend on absorption, inactivation, and excretion rates of the drug as well as the delivery rate of the compound from the formulation, and will be suitable for short or long term use for the treatment of acute or chronic conditions, respectively. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of ordinary skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the antihistamine formulation and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determine the optimum time and/or amount of administration, which will require no more than routine experimentation consisting of monitoring the subject and adjusting the dosage and/or timing.

The combined use of several NSAIDs formulated into the compositions of the present invention may reduce the required dosage for any individual component because the onset and duration of effect of the different components may be complimentary. In such combined therapy, the different active agents (i.e., the one antistamine agent) may be delivered together or separately, and simultaneously or at different times within the day. Additionally, the combination of other vasculature modifying agents such as beta blockers and adrenergic activators may increase efficacy of the topical ophthalmic solution in the treatment of acute and prophylaxis of migraine.

Efficacy of the formulations and compositions of the invention in prophylaxis and acute treatment of the signs and symptoms associated with migraine headache have previously been assessed by the following clinical endpoints. Acute treatments for migraine have used the following regulatory endpoints: 4 & 5 point headache pain (severity scale) at 2 hrs & 4 hrs compared to placebo, reduction of phonophobia/photophobia at 2 hr & 4 hr compared to placebo, usage of rescue medication after first dose compared to placebo (Migranal), headache response up to 2 hrs post dose compared to placebo, reduction in nausea/phonophobia/photophobia compared to placebo, percentage of patients taking a 2^{nd} dose after 2 hrs (hours) post dose compared to placebo (Imitrex nasal topical), relief of headache pain at 1 hr & 2 hrs post injection compared to placebo, reduction of phonophobia/photophobia/nausea/vomiting at 2 hr & 4 hr compared to placebo (Imitrex Subcutaneous Injection). Treatments for the prophylaxis of migraine headache: change from baseline in frequency of headache from week 4 to week 24 post treatment compared to placebo, change from baseline in total cumulative hours of headache on headache days from week 4 to week 24 post treatment compared to placebo (Botox Injection), reduction in mean 4 week headache rates in the 8 week treatment phase after the 4 week placebo phase compared to placebo (Depakote), reduction in the headache index unit (a composite of the number of days with headache and associated severity of headache) compared to placebo (Inderal), reduction in mean 4 week headache rates in the 8 week treatment phase after the 4 week placebo phase compared to placebo (Stavzor), reduction in the change of 4 week migraine rate throughout the 26 week study compared to placebo (Topamax)

### Packaging

The formulations of the present invention may be packaged as either a single dose product or a multi-dose product. The single dose product is sterile prior to opening of the package and all of the composition in the package is intended to be used in a single application to one or both eyes of a patient. The use of an antimicrobial preservative to maintain the sterility of the composition after the package is opened is generally unnecessary. The formulations, if an ointment formulation, may be packaged as appropriate for an ointment, as is known to one of skill in the art.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and other publications by the Food and Drug Administration, and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

Ophthalmic ointments may be produced as follows: if necessary, antiseptics, surfactants, stabilizers, alcohols, esters or oils are blended with an ointment base such as liquid paraffin or white petrolatum placed in a mortar or a mixing machine for ointment to form a mixture. This is followed by addition of the active ingredients and the resulting mixture is mixed until uniform and kneaded to form the ophthalmic ointment. The ointment thus prepared is filled into a bottle or tube for ointment to obtain the ophthalmic ointment containing the active ingredients of the present invention.

### EXAMPLES

The invention now being generally described, it will be more readily understood by reference to the following examples which are included merely for purposes of illustration of certain aspects and embodiments of the present invention, and are not intended to limit the invention in any way.

### Example 1

Data from two subjects has shown that instillation of one drop of timolol 0.5% and one drop of ketotifen 0.05% in the eye was successful in providing relief from migraine attack.

### Example 2

Data from two subjects with occasional migraine has shown that one drop of ketotifen 0.025% in the eye was successful in providing relief within 2 hours post treatment

### Example 3

Data from two subjects with occasional migraine has shown that one drop of ketotifen 0.05% in the eye was successful in providing relief within 2 hours post treatment

### REFERENCES

1. Zhang X C, Strassman A M, Burstein R, Levy D. (2007) Sensitization and Activation of Intracranial Meningeal Nociceptors by Mast Cell Mediators. J Pharmacol Exp Ther. August; 322(2):806-812.

2. Haimart M, Pradalier A, Launay J M, Dreux C, Dry J. (1987) Whole blood and plasma histamine in common migraine. Cephalalgia 7:39-42.

3. Heatley R V, Denburg J A, Bayer N, Bienenstock J. (1982) Increased plasma histamine levels in migraine patients. Clin Allergy 12:145-149.

4. Lewis D W, Diamond S, Scott D, Jones V. (2004) Prophylactic treatment of pediatric migraine. Headache 44:230-237.

5. Togha M, Ashrafian H, Tajik P. (2006) Open-label trial of cinnarizine in migraine prophylaxis. Headache 46:498-502.

6. Goodman & Gilman's The Pharmacological Basis of Therapeutics, 11th Edition, Brunton L,L; Editor-in-chief. Copyright 2006. The McGraw-Hill Companies, Inc.

7. Histamine, Serotonin and the Ergot Alkaloids. Lange's Basic and Clinical Pharmacology. Copyright 2007, The McGraw-Hill Companies, Inc. Theoharides, T.,C. et al. (2005) The role of mast cells in migraine pathophysiology. Brian Research Reviews 49:65-76

8. Martin V.,T., et al (2011) Allergy and Immunotherapy: Are They Related to Migraine Headache? Headache 51:8-20

9. Levy, D. et al. (2007) Mast cell degranulation activates a pain pathway underlying migraine headache Pain. 130(1-2): 166-176.

10. Levy, D. (2009) Migraine Pain, Meningeal Inflammation, and Mast Cells Current Pain & Headache Reports 13:237-240

11. Ba'albaki, H. & Rapoport, A. (2008) Mast Cells Activate the Renin Angiotensin System and Contribute to Migraine: A Hypothesis Headache 48:1499-1505

12. Mansfield, L. E.

13. Examining the Utilization and Tolerability of the Non-Sedating Antihistamine Levocetirizine in England Using Prescription-Event Monitoring Data Drug Self 34(12):1177-1189

14. Lewis, D & Brenner, M. (2008) The Treatment of Migraine Headaches in Children and Adolescents

J Pediatr Pharmacol Ther 13:17-24

1. A topical ophthalmic formulation comprising at least one antihistamine agent, in an effective amount to treat or prevent the signs and symptoms of migraine headache.

2. The formulation of claim 1, wherein the antihistamine agent is a mast cell stabilizer.

3. The formulation of claim 2, wherein the anti-histamine or anti-histamine/mast cell stabilizer is ketotifen fumarate, epinastine, bepotastine, cetirizine, olopatadine hydrochloride, alcaftadine, norketotifen, azelastine chlorpheniramine, brompheniramine, diphenhydramine, clemastine, hydroxyzine, chlorpheniramine, doxylamine, alimemazine, phenyltoloxamine, promethazine, mepyramine, cyproheptadine, hydroxyzine, antazoline, tripelennamine, orphenadrine, bromazine, clemastine, dimetindene, azatadine, loratadine, fexofenadine, desloratadine, levocetirizine, carbinoxamine, triprolidine, dexchlorpheniramine, pheniramine, ebastine, dexbrompheniramine, astemizole, rupatadine, mizolastine, acrivastine, bilastine, terfenadine, quifenadine, or levocabastine, derivatives thereof, and pharmacologically active salts thereof.

4. The formulation of claim 3, wherein the anti-histamine/mast cell stabilizer is ketotifen fumarate.

5. The formulation of claim 4, wherein the concentration of the ketotifen is about 0.025-0.05%.

6. The formulation of claim 3, wherein the anti-histamine/mast cell stabilizer is alcaftadine.

7. The formulation of claim 6, wherein the concentration of the alcaftadine is about 0.025-0.5%.

8. An ophthalmic formulation of claim 1, wherein the migraine headache is with or without aura.

9. An ophthalmic formulation of claim 1, wherein the frequency, severity and duration of the migraine headache is reduced.

10. The ophthalmic formulation of claim 1, further comprising one or more vasculature modifying agents.

11. The ophthalmic formulation of claim 10, wherein the vasculature modifying agent is a β adrenergic receptor antagonist.

12. The ophthalmic formulation of claim 11, wherein the β adrenergic receptor antagonist is also an H-1 antagonist.

13. The ophthalmic formulation of claim 12 wherein the H-1 antagonist is bisoprolol, timolol, propranolol, nadolol, metoprolol, metipranolol, nebivolol, or betaxolol.

14. The ophthalmic formulation of claim 11, wherein the antihistamine is ketotifen fumarate and the β adrenergic receptor antagonist is timolol maleate.

15. The ophthalmic formulation of claim 12, wherein the concentration of ketotifen fumarate is about 0.025-0.05% and the concentration of timolol maleate is about 0.25-0.5%.

16. The ophthalmic formulation of claim 11, wherein the antihistamine is alcaftadine and the β adrenergic receptor antagonist is timolol maleate.

17. The ophthalmic formulation of claim 16, wherein the concentration of alcaftadine is about 0.025-0.5% and the concentration of timolol maleate is about 0.25-0.5%.

18. A method for treating and/or preventing the signs and symptoms of migraine headache comprising administering the topical ophthalmic formulation of claim 1.

19-22. (canceled)

23. A method for treating and/or preventing the signs and symptoms of migraine headache comprising administering a topical ophthalmic formulation comprising: at least one antihistamine agent, in an amount effective to treat or prevent the signs and symptoms of migraine headache; and, one or more vasculature modifying agents.

24. The method of claim 23, wherein the vasculature modifying agent is a β adrenergic receptor antagonist.

25. The method of claim 24, wherein the antihistamine agent is ketotifen fumarate and the β adrenergic receptor antagonist is timolol maleate.

26. The method of claim 25 wherein the concentration of ketotifen fumarate is about 0.025-0.05% and the concentration of timolol maleate is about 0.25-0.5%.

27. The method of claim 24 wherein the antihistamine agent is alcaftadine and the β adrenergic receptor antagonist is timolol maleate.

28. The method of claim 27 wherein the concentration of alcaftadine is about 0.025-0.5% and the concentration of timolol maleate is about 0.25-0.5%.
